(19) United States
(12) Patent Application Publication
(10) Pub. No.: US 2021/0045922 A1
(43) Pub. Date: Feb. 18, 2021

(54) Sleep Mask Assembly

(71) Applicant: Cheryl Lockett, Indianapolis, IN (US)

(72) Inventor: Cheryl Lockett, Indianapolis, IN (US)

(21) Appl. No.: 16/539,546

(22) Filed: Aug. 13, 2019

(51) Int. Cl. A61F 9/04; A61F 11/12

(52) U.S. Cl. CPC A61F 9/04; A61M 21/02; A61F 11/12

(57) ABSTRACT

A sleep mask assembly includes a mask that is worn on a user's face when the user is sleeping. The mask has a compartment that is integrated therein. A pad is insertable into the compartment in the mask and the pad is comprised of a thermally conductive material. In this way the pad can be cooled to reduce swelling on the user's face when the mask is worn and the pad is inserted into the compartment. A band is coupled to the mask and the band is worn around the user's head. A pair of earphones is provided and each of the earphones is coupled to the band. Each of the earphones can be inserted into a respective one of the user's ears when the mask is worn.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to mask device and more particularly pertains to a new mask device for wearing during sleep and facilitating thermal therapy.

### BRIEF SUMMARY OF THE INVENTION

An embodiment of the disclosure meets the needs presented above by generally comprising a mask that is worn on a user's face when the user is sleeping. The mask has a compartment that is integrated therein. A pad is insertable into the compartment in the mask and the pad is comprised of a thermally conductive material. In this way the pad can be cooled to reduce swelling on the user's face when the mask is worn and the pad is inserted into the compartment. A band is coupled to the mask and the band is worn around the user's head. A pair of earphones is provided and each of the earphones is coupled to the band. Each of the earphones can be inserted into a respective one of the user's ears when the mask is worn.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a sleep mask assembly according to an embodiment of the disclosure.

FIG. 2 is a perspective in-use view of an embodiment of the disclosure.

FIG. 3 is a left side phantom view of an embodiment of the disclosure.

FIG. 4 is a front phantom view of an embodiment of the disclosure.

FIG. 5 is a back exploded view of an embodiment of the disclosure.

FIG. 6 is a cross sectional view taken along line 6-6 of FIG. 4 of an embodiment of the disclosure.

FIG. 7 is a perspective phantom view of an embodiment of the disclosure.

### DETAILED DESCRIPTION OF THE INVENTION

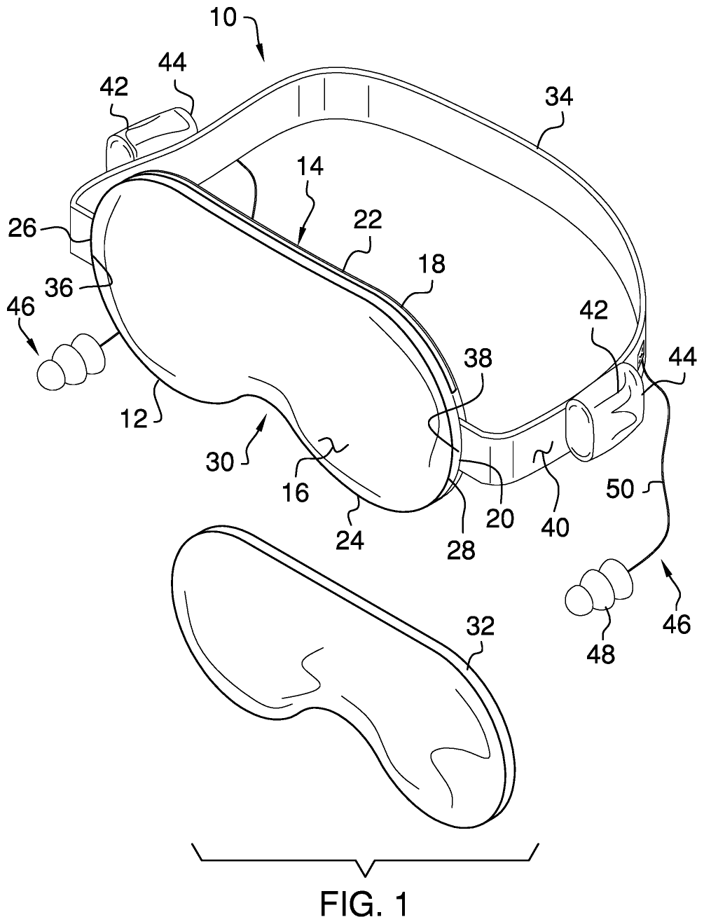

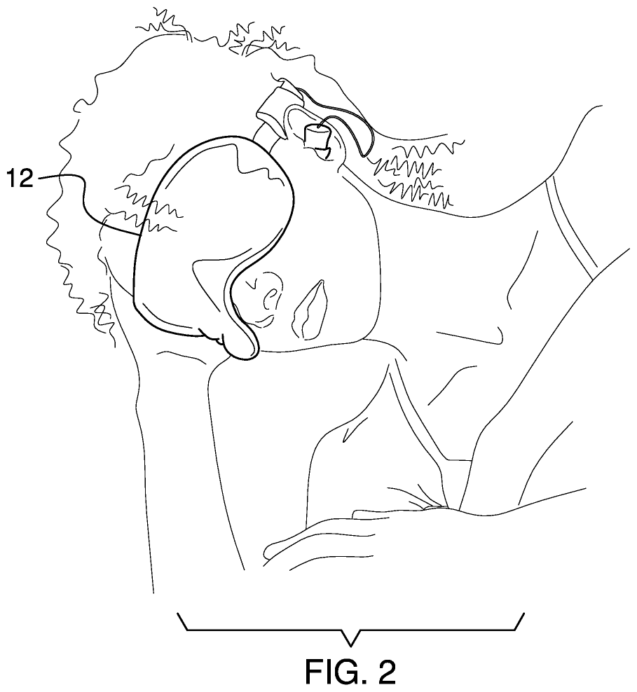

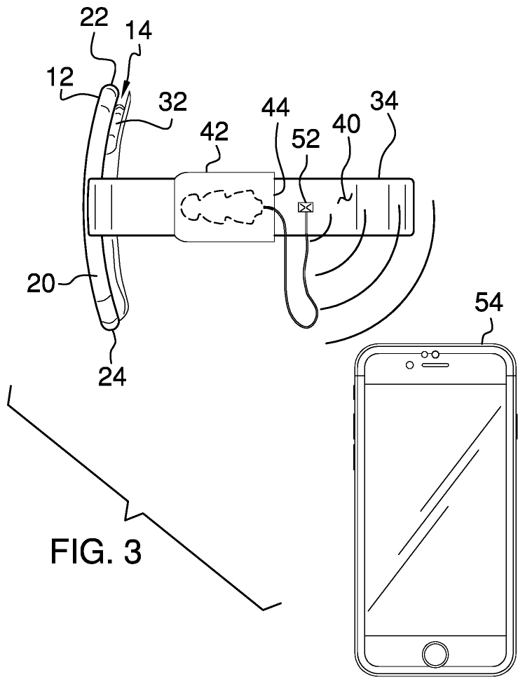

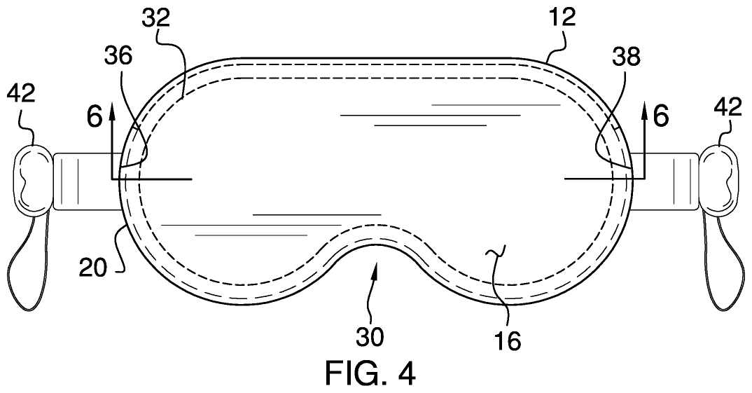

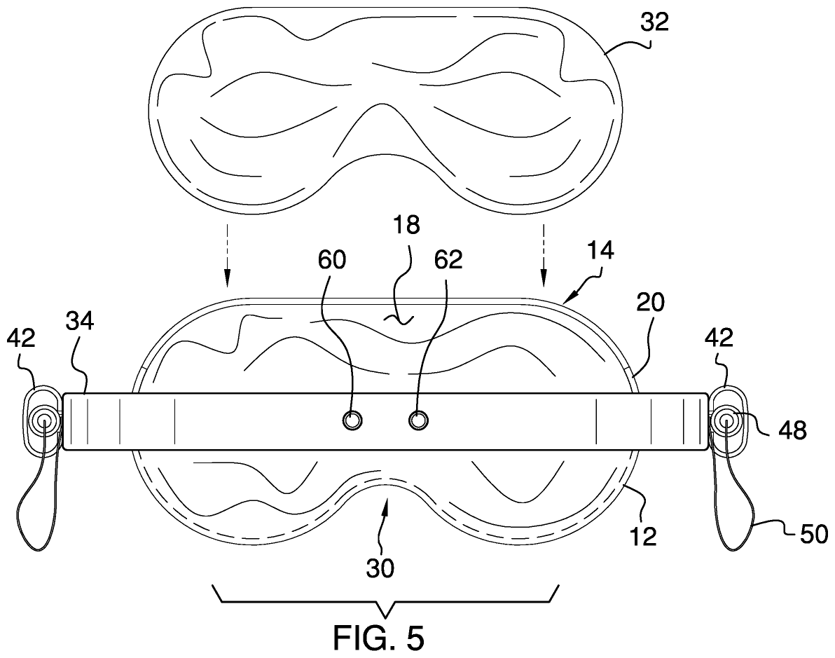

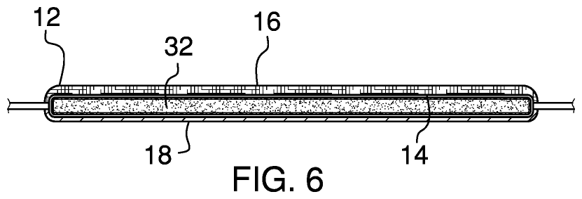

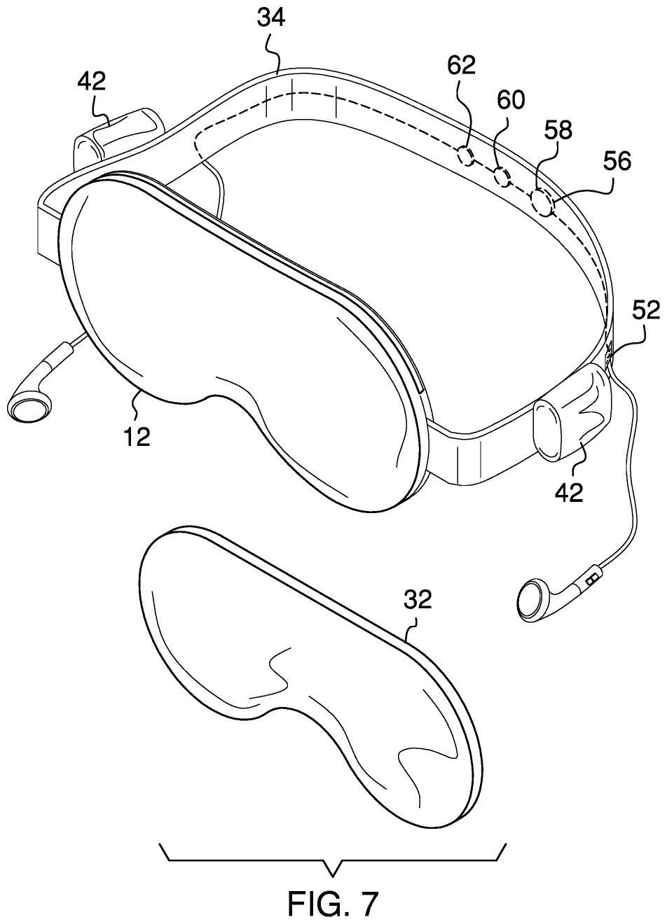

With reference now to the drawings, and in particular to FIGS. 1 through 7 thereof, a new mask device embodying the principles and concepts of an embodiment of the disclosure and generally designated by the reference numeral 10 will be described.

As best illustrated in FIGS. 1 through 7, the sleep mask assembly 10 generally comprises a mask 12 that is worn on a user's face when the user is sleeping. The mask 12 has a compartment 14 that is integrated therein. Additionally, the mask 12 is comprised of a deformable and thermally conductive material. The mask 12 has a front surface 16, a back surface 18 and a perimeter edge 20 extending therebetween. The perimeter edge 20 has a top side 22, a bottom side 24, a first lateral side 26 and a second lateral side 28. The mask 12 is elongated between the first lateral side 26 and the second lateral side 28.

The back surface 18 rests against the user's face when the mask 12 is worn. The bottom side 24 has a nose slot 30 extending upwardly toward the top side 22 to accommodate the user's nose when the mask 12 is worn having the mask 12 extending across the user's eyes. The compartment 14 extends through the top side 22 downwardly toward the bottom side 24. Additionally, the compartment 14 is positioned between the front surface 16 and the back surface 18.

A pad 32 is provided and the pad 32 is insertable into the compartment 14 in the mask 12. The pad 32 is comprised of a thermally conductive material, such as a thermal gel or the like. Thus, the pad 32 can be cooled to reduce swelling on the user's face when the mask 12 is worn and the pad 32 is inserted into the compartment 14. Additionally, the pad 32 can be warmed to facilitate heat therapy when the mask 12 is worn and the pad 32 is inserted into the compartment 14.

A band 34 is coupled to the mask 12 wherein the band 34 is configured to be worn around the user's head. The band 34 has a first end 36, a second end 38 and a first surface 40 extending therebetween. The first end 36 is coupled to the first lateral side 26 of the perimeter edge 20 of the mask 12. The second end 38 is coupled to the second lateral side 28 of the perimeter edge 20 of the mask 12. Moreover, the band 34 may be comprised of an elastomeric material for retaining the mask 12 against the user's face.

A pair of pouches 42 is provided and each of the pouches 42 is coupled to the band 34. Each of the pouches 42 is positioned on the first surface 40 of the band 34 and each of the pouches 42 is positioned adjacent to a respective one of the first end 36 and the second end 38 of the band 34. Each of the pouches 42 has an open end 44 to access an interior of the pouches 42.

A pair of earphones 46 is provided and each of the earphones 46 is coupled to the band 34. Each of the earphones 46 can be inserted into a respective one of the user's ears when the mask 12 is worn. Each of the earphones 46 is inserted into a respective one of the pouches 42 for storage. Additionally, each of the earphones 46 includes an earbud 48 and a conductor 50 that is coupled between the earbud 48 and the band 34. The earbud 48 emits audible sound into the user's ear and the earbud 48 may include an electronic speaker.

A receiver 52 is coupled to the band 34 and the receiver 52 has the conductor 50 of each of the earphones 46 being electrically coupled thereto. The receiver 52 is in wireless communication with an extrinsic electronic device 54 for receiving an audio signal from the extrinsic electronic device 54. In this way each of the earbuds 48 can receive the audio signal. The receiver 52 may comprise a radio frequency receiver and the receiver 52 may employ Bluetooth communication protocols. Moreover, the extrinsic electronic device 54 may be a smart phone, a personal computer or any other type of data storage device.

A power supply 56 is coupled to the band 34, the power supply 56 is electrically coupled to the receiver 52 and the power supply 56 comprises at least one battery 58. A power button 60 is coupled to the band 34 and the power button 60 is electrically coupled to the receiver 52 for tuning the receiver 52 on and off. Additionally, a connect button 62 is coupled to the band 34 and the connect button 62 is electrically coupled to the receiver 52. The connect button 62 places the receiver 52 in communication with the extrinsic electronic device 54.

In use, the pad 32 is either cooled or warmed, depending on the user's preference for cooling therapy or heat therapy. The pad 32 is inserted into the compartment 14 in the mask 12 and the mask 12 is worn on the user's face. In this way the pad 32 is placed in thermal communication with the mask 12, and subsequently the user's face. The receiver 52 can be placed in electrical communication with the extrinsic electronic device 54. In this way the earbuds 48 can emit audible sounds into the user's ears when the earbuds 48 are inserted into the user's ears.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sleep mask assembly being configured to be worn on a user's face while sleeping, said assembly comprising:
a mask being worn on a user's face when the user is sleeping, said mask having a compartment being integrated therein;
a pad being insertable into said compartment in said mask, said pad being comprised of a thermally conductive material thereby facilitating said pad to be cooled wherein said pad is configured to reduce swelling on the user's face when said mask is worn and said pad is inserted into said compartment;
a band being coupled to said mask wherein said band is configured to be worn around the user's head;
a pair of pouches, each of said pouches being coupled to said band; and
a pair of earphones, each of said earphones being coupled to said band wherein each of said earphones is configured to be inserted into a respective one of the user's ears when said mask is worn, each of said earphones being inserted into a respective one of said pouches for storage.

2. The assembly according to claim 1, wherein said mask has a front surface, a back surface and a perimeter edge extending therebetween, said perimeter edge having a top side, a bottom side, a first lateral side and a second lateral side, said mask being elongated between said first lateral side and said second lateral side, said back surface resting against the user's face when said mask is worn, said bottom side having a nose slot extending upwardly toward said top side wherein said nose slot is configured to accommodate the user's nose when said mask is worn having said mask extending across the user's eyes.

3. The assembly according to claim 2, wherein said compartment extends through said top side downwardly toward said bottom side, said compartment being positioned between said front surface and said back surface.

4. The assembly according to claim 2, wherein said band has a first end, a second end and a first surface extending therebetween, said first end being coupled to said first lateral side of said perimeter edge of said mask, said second end being coupled to said second lateral side of said perimeter edge of said mask.

5. The assembly according to claim 4, wherein each of said pouches is positioned on said first surface of said band, each of said pouches being positioned adjacent to a respective one of said first end and said second end of said band, each of said pouches having an open end to access an interior of said pouches.

6. The assembly according to claim 1, wherein each of said earphones includes an earbud and a conductor being coupled between said earbud and said band wherein said earbud is configured to emit audible sound into the user's ear.

7. The assembly according to claim 6, further comprising a receiver being coupled to said band, said receiver having said conductor of each of said earphones being electrically coupled thereto, said receiver being in wireless communication with an extrinsic electronic device for receiving an audio signal from the extrinsic electronic device wherein each of said earbuds is configured to receive the audio signal.

8. The assembly according to claim 7, further comprising a power supply being coupled to said band, said power supply being electrically coupled to said receiver, said power supply comprising at least one battery.

9. A sleep mask assembly being configured to be worn on a user's face while sleeping, said assembly comprising:
a mask being worn on a user's face when the user is sleeping, said mask having a compartment being integrated therein, said mask having a front surface, a back surface and a perimeter edge extending therebetween, said perimeter edge having a top side, a bottom side, a first lateral side and a second lateral side, said mask being elongated between said first lateral side and said second lateral side, said back surface resting against the user's face when said mask is worn, said bottom side having a nose slot extending upwardly toward said top side wherein said nose slot is configured to accommodate the user's nose when said mask is worn having said mask extending across the user's eyes, said compartment extending through said top side downwardly toward said bottom side, said compartment being positioned between said front surface and said back surface;
a pad being insertable into said compartment in said mask, said pad being comprised of a thermally conductive material thereby facilitating said pad to be cooled wherein said pad is configured to reduce swelling on the user's face when said mask is worn and said pad is inserted into said compartment;
a band being coupled to said mask wherein said band is configured to be worn around the user's head, said band having a first end, a second end and a first surface extending therebetween, said first end being coupled to said first lateral side of said perimeter edge of said mask, said second end being coupled to said second lateral side of said perimeter edge of said mask;
a pair of pouches, each of said pouches being coupled to said band, each of said pouches being positioned on said first surface of said band, each of said pouches being positioned adjacent to a respective one of said first end and said second end of said band, each of said pouches having an open end to access an interior of said pouches;
a pair of earphones, each of said earphones being coupled to said band wherein each of said earphones is configured to be inserted into a respective one of the user's ears when said mask is worn, each of said earphones being inserted into a respective one of said pouches for storage, each of said earphones including an earbud and a conductor being coupled between said earbud and said band wherein said earbud is configured to emit audible sound into the user's ear;
a receiver being coupled to said band, said receiver having said conductor of each of said earphones being electrically coupled thereto, said receiver being in wireless communication with an extrinsic electronic device for receiving an audio signal from the extrinsic electronic device wherein each of said earbuds is configured to receive the audio signal; and
a power supply being coupled to said band, said power supply being electrically coupled to said receiver, said power supply comprising at least one battery.
